(19) United States
(12) Patent Application Publication
(10) Pub. No.: US 2008/0076119 A9
(43) Pub. Date: Mar. 27, 2008

(54) Composite organic inorganic nanoclusters

(76) Inventors: Lei Sun, Santa Clara, CA (US); Xing Su, Cupertino, CA (US); Mineo Yamakawa, Campbell, CA (US); Zhang Jingwu, San Jose, CA (US); Narayan Sundararajan, San Francisco, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/081,772

(22) Filed: Mar. 15, 2005

Related U.S. Application Data
(63) Continuation-in-part of application No. 10/940,698, filed on Sep. 13, 2004.
Application No. 10/940,698 is a continuation-in-part of application No. 10/916,710, filed on Aug. 11, 2004.
Application No. 10/916,710 is a continuation-in-part of application No. 10/830,422, filed on Apr. 21, 2004, now abandoned.
Application No. 10/830,422 is a continuation-in-part of application No. 10/748,336, filed on Dec. 29, 2003, now abandoned.
(63) Continuation-in-part of application No. 11/021,682, filed on Dec. 23, 2004.
Application No. 11/021,682 is a continuation-in-part of application No. 10/830,422, filed on Apr. 21, 2004, now abandoned.
Application No. 10/830,422 is a continuation-in-part of application No. 10/748,336, filed on Dec. 29, 2003, now abandoned.

(51) Int. Cl. C12Q 1/68; G01N 33/53

(52) U.S. Cl. 435/6; 435/7.1; 977/900; 977/924

(57) ABSTRACT

Metallic nanoclusters capable of providing an enhanced Raman signal from an organic Raman-active molecule incorporated therein are provided. The nanoclusters may be further functionalized, for example, with coatings and layers, such as adsorption layers, metal coatings, silica coatings, probes, and organic layers. The nanoclusters are generally referred to as COINs (composite organic inorganic nanoparticles) and are capable of acting as sensitive reporters for analyte detection. A variety of organic Raman-active compounds and mixtures of compounds can be incorporated into the nanocluster.

### BACKGROUND OF THE

1. Field of the Invention

Embodiments of the present invention relate generally to metallic nanoclusters having organic compounds incorporated therein.

2. Background Information

The ability to detect and identify trace quantities of analytes has become increasingly important in many scientific disciplines, ranging from part per billion analyses of pollutants in sub-surface water to analysis of treatment drugs and metabolites in blood serum. Additionally, the ability to perform assays in multiplex fashion greatly enhances the rate at which information can be acquired. Devices and methods that accelerate the processes of elucidating the causes of disease, creating predictive and or diagnostic assays, and developing effective therapeutic treatments are valuable scientific tools. A principle challenge is to develop an identification system for a large probe set that has distinguishable components for each individual probe.

Among the many analytical techniques that can be used for chemical analyses, surface-enhanced Raman spectroscopy (SERS) has proven to be a sensitive method. A Raman spectrum, similar to an infrared spectrum, consists of a wavelength distribution of bands corresponding to molecular vibrations specific to the sample being analyzed (the analyte). Raman spectroscopy probes vibrational modes of a molecule and the resulting spectrum, similar to an infrared spectrum, is fingerprint-like in nature. As compared to the fluorescent spectrum of a molecule which normally has a single peak exhibiting a half peak width of tens of nanometers to hundreds of nanometers, a Raman spectrum has multiple structure-related peaks with half peak widths as small as a few nanometers.

To obtain a Raman spectrum, typically a beam from a light source, such as a laser, is focused on the sample generating inelastically scattered radiation which is optically collected and directed into a wavelength-dispersive spectrometer. Although Raman scattering is a relatively low probability event, SERS can be used to enhance signal intensity in the resulting vibrational spectrum. Enhancement techniques make it possible to obtain a 10⁶ to 10¹⁴ fold Raman signal enhancement.

A prerequisite for multiplex analyses of a complex sample is to have a coding system that possesses identifiers for a large number of analytes in the sample. Additionally, the identifiers, or reporters, for analyte detection may need to possess different properties depending on a user-selected application, such as for example, mechanical and/or chemical stability. Depending on the intended use, reporters may need to be chemically compatible with diverse applications, such as biochemical analyses, and be capable of being stably functionalized over a variety of conditions with probes that allow for the complexation of the identifier with an analyte in a sample.

### BRIEF DESCRIPTION OF THE FIGURES

The following drawings are included to further demonstrate certain aspects of the disclosed embodiments of the invention.

FIGS. 1A and B show Raman spectra obtained from COINs (composite organic inorganic nanoclusters) that incorporate a single type of Raman label and three different Raman labels, respectively. (Key: 8-aza-adenine (AA), 9-aminoacridine (AN), methylene blue (MB).) Representative peaks are indicated by arrows; peak intensities have been normalized to respective maximums; the Y axis values are in arbitrary units; spectra are offset by 1 unit from each other.

FIGS. 2A and B show signatures of COINs with double and triple Raman labels. The 3 Raman labels used were 8-aza-adenine (AA), 9-aminoacridine (AN), and methylene blue (MB). The main peak positions are indicated by arrows; the peak heights (in arbitrary units) were normalized to respective maximums; spectra are offset by 1 unit from each other.

FIG. 3 illustrates a structure of an organic molecule that can be used as a Raman label.

FIG. 4 illustrates a comparison of COIN Raman intensities for ten Raman labels.

FIGS. 5A, B, C, and D shows Raman spectra from several organic Raman labels and COINs.

FIG. 6 is a-schematic illustrating a use of COINs as reporters for analyte detection.

FIG. 7 illustrates a use of COINs as tags for analyte detection in solution in which a protein analyte is detected according to an intrinsic Raman signal from a bound COIN.

FIG. 8 illustrates a use of COINs as tags for cell-surface antigen identification. A sample containing a cell having various surface antigens is contacted with a COIN having attached antibodies specific for a known cell-surface antigen. The. COIN attaches specifically to the known antigen. The cell is stained with a fluorescent dye. The cell is counted using fluorescent-based cell counting techniques, and the intrinsic Raman signal from the COIN is collected. The fluorescence signal is correlated with the Raman signal to determine the presence of the target cellular analyte in the sample.

### DETAILED DESCRIPTION OF THE INVENTION

As described more fully herein, composite organic inorganic nanoclusters (COINs) are composed of a metal and at least one organic Raman-active compound. Interactions between the metal of the clusters and the Raman-active compound(s) enhance the Raman signal obtained from the Raman-active compound(s) when the nanoparticle is excited by a laser. COINs according to embodiments of the present invention can perform as sensitive reporters for use in analyte detection. Since a large variety of organic Raman-active compounds can be incorporated into the nanoclusters, a set of COINs can be created in which each member of the set has a Raman signature unique to the set. Thus, COINs can also function as sensitive reporters for highly parallel analyte detection. Furthermore, not only are the intrinsic enhanced Raman signatures of the nanoparticles of the present invention sensitive reporters, but sensitivity may also be further enhanced by incorporating thousands of Raman labels into a single nanocluster and or attaching multiple nanoclusters to a single analyte.

It was found that aggregated metal colloids fused at elevated temperature and that organic Raman labels could be incorporated into the coalescing metal particles. These coalesced metal particles formed stable clusters and produced intrinsically enhanced Raman scattering signals for the incorporated organic label(s). Thus, the COINs of the present invention do not require an amplification step to function as sensitive reporters for analyte detection since Raman enhancement is intrinsic in the cluster.

The interaction between the organic Raman label molecules and the metal colloids has mutual benefits. Besides serving as signal sources, the organic molecules induce a metal particle association that is in favor of electromagnetic signal enhancement. Additionally, the internal nanocluster structure provides spaces to hold Raman label molecules, especially in the junctions between the metal particles that make up the cluster. In fact, it is believed that the strongest enhancement is achieved from the organic molecules located in the junctions between the metal particles of the nanoclusters.

The nanoclusters can be prepared using standard metal colloid chemistry. Generally, the nanoclusters are less than 1 μm in size, and are formed by particle growth in the presence of organic compounds. The preparation of such nanoparticles also takes advantage of the ability of metals to adsorb organic compounds. Indeed, since Raman-active organic compounds are adsorbed onto the metal cluster during formation of the metallic colloids, many Raman-active organic compounds can be incorporated into a nanoparticle.

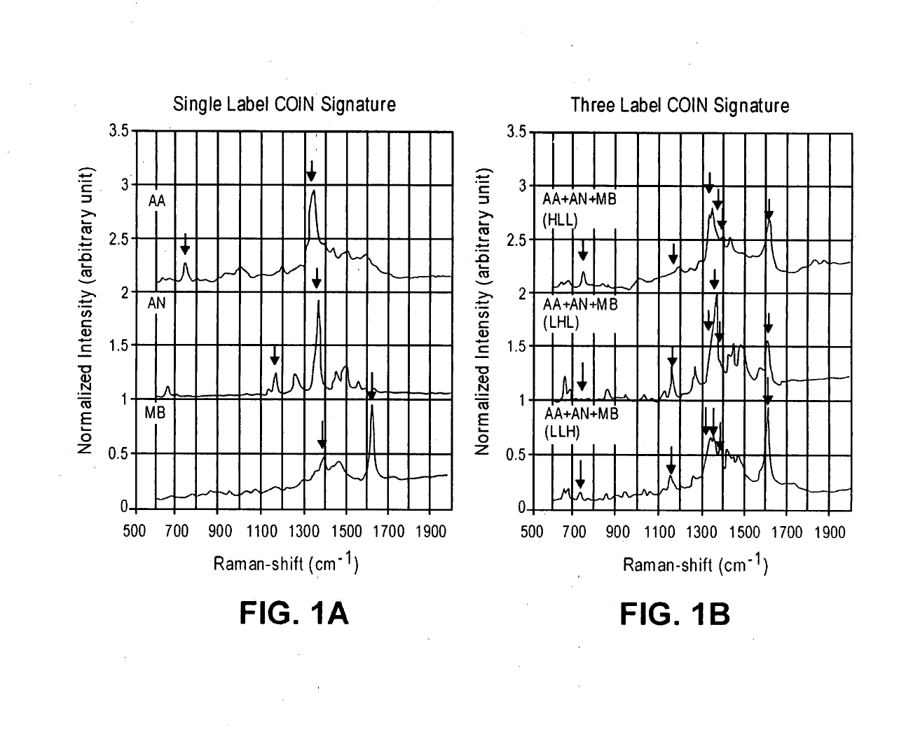

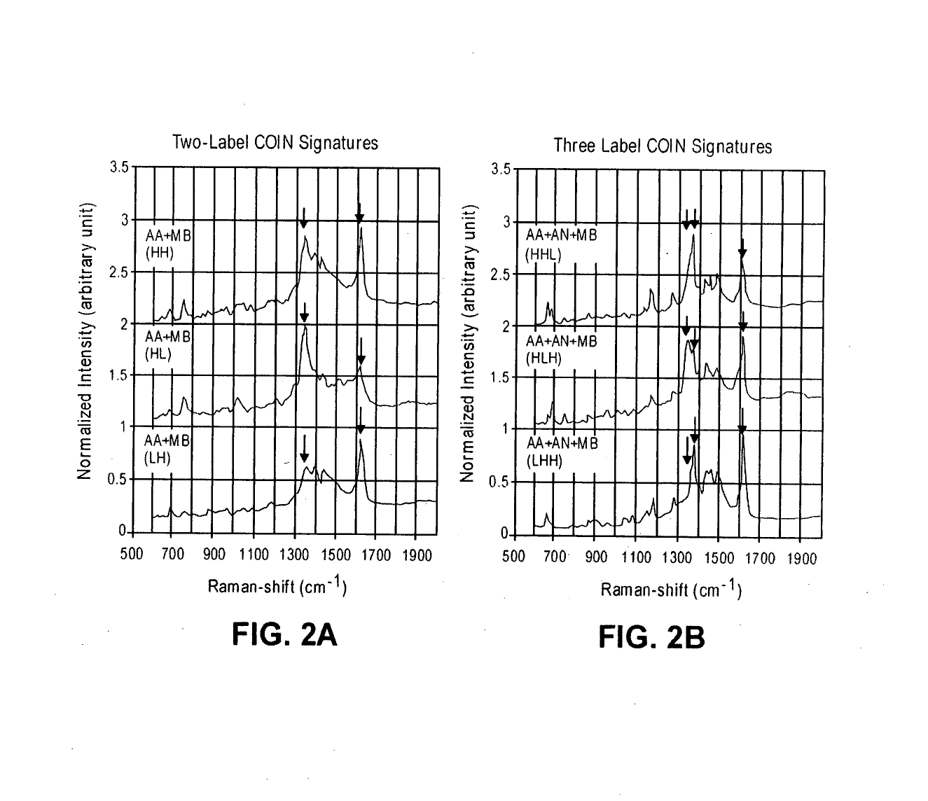

Not only can COINs be synthesized with different Raman labels, but COINs may also be created having different mixtures of Raman labels and also different ratios of Raman labels within the mixtures. Referring now to FIGS. 1 and 2, FIG. 1A shows signatures of COINs made with a single Raman-active organic compound, demonstrating that each Raman-active organic compound produced a unique signature. FIG. 1B shows signatures of COINs made with mixtures of three Raman labels at concentrations that produced signatures as indicated: HLL means high peak intensity for 8-aza-adenine (AA) (H) and low peak intensity for both 9-aminoacridine (AN) (L) and methylene blue (MB) (L); LHL means low peak intensity for AA (L), high peak intensity for AN (H) and low for MB (L); LLH means low for both AA (L) and AN (L) and high for MB (H). COINs in these examples were made with individual or mixtures of Raman labels at concentrations from 2.5 μM to 20 μM, depending on the signature desired. Peak heights can be adjusted by varying label concentrations, but they might not be proportional to the concentrations of the labels used due to different absorption affinities of the Raman labels for the metal surfaces. FIG. 2A shows signatures of COINs made with 2 Raman labels (AA and MB) at concentrations designed to achieve the following relative peak heights: AA=MB (HH), AA>MA (HL), and AA<MB (LH). FIG. 2B shows Raman signatures of COINs made from mixtures of the 3 Raman labels at concentrations that produced the following signatures: HHL means high peak intensities for AA (H) and AN (H) and low peak intensity for MB (L); HLH means high peak intensity for AA (H), low peak intensity for AN (L), and high peak intensity for MB (H); and LLH means low peak intensities for AA (L) and AN (L), and high peak intensity for MB (H). COINs in these examples were made by the oven incubation procedure with mixtures of 2 or 3 Raman labels at concentrations from 2.5 to 20 μM, depending on the signatures desired. Thus, it is possible to create a large number of different molecular identifiers using the COINs of the present invention. Theoretically, over a million COIN signatures could be made within the Raman shift range of 500-2000 cm⁻¹.

Table 1 provides examples of the types of organic compounds that can be used to build COINs. In general, Raman-active organic compound refers to an organic molecule that produces a unique SERS signature in response to excitation by a laser. Typically the Raman-active compound has a molecular weight less than about 500 Daltons.

TABLE 1

| No. | Abbreviation | Name | Structure |
|---|---|---|---|
| 1 | AAD (AA) | 8-Aza-adenine | 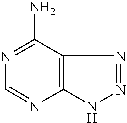 |
| 2 | BZA (BA) | N-Benzoyladenine | 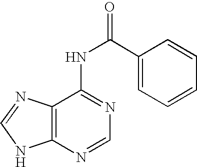 |
| 3 | MBI | 2-Mercapto-benzimidazole | 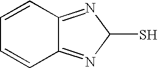 |
| 4 | APP | 4-Amino-pyrazolo[3,4-d]pyrimidine | 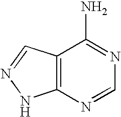 |
| 5 | ZEN | Zeatin | 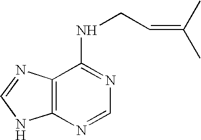 |
| 6 | MBL (MB) | Methylene Blue | 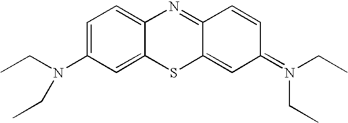 |
| 7 | AMA (AN, AM) | 9-Amino-acridine | 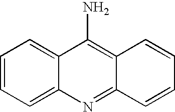 |
| 8 | EBR | Ethidium Bromide | 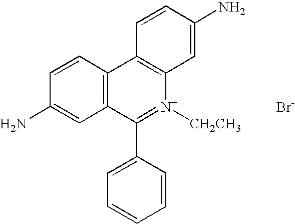 |
| 9 | BMB | Bismarck Brown Y | 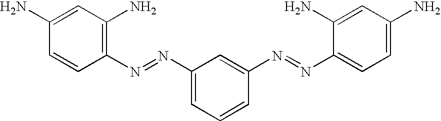 |
| 10 | NBA | N-Benzyl-aminopurine | 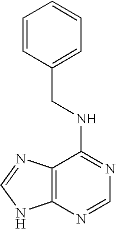 |
| 11 | THN | Thionin acetate | 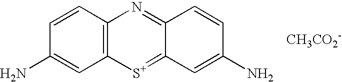 |
| 12 | DAH | 3,6-Diaminoacridine | 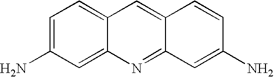 |
| 13 | CYP | 6-Cyanopurine | 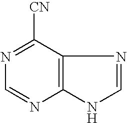 |
| 14 | AIC | 4-Amino-5-imidazole-carboxamide hydrochloride | 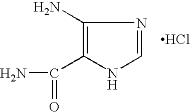 |
| 15 | DII | 1,3-Diiminoisoindoline | 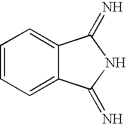 |
| 16 | R6G | Rhodamine 6G | 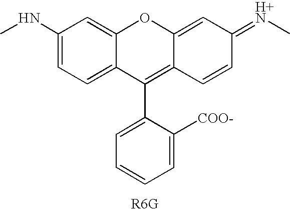 |
| 17 | CRV | Crystal Violet | 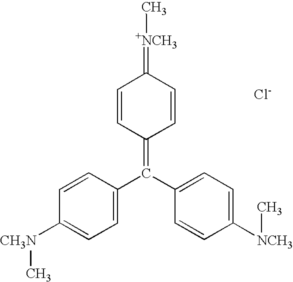 |
| 18 | BFU | Basic Fuchsin | 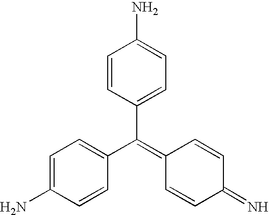 |
| 19 | ANB | Aniline Blue Diammonium salt | 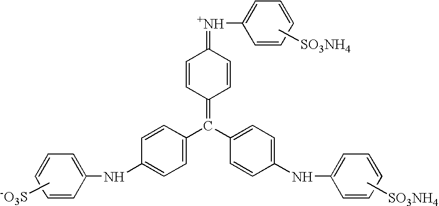 |
| 20 | ACA | N-[(3-(Anilinomethylene)-2-chloro- 1-cyclohexen-1- yl)methylene]aniline monohydrochloride | 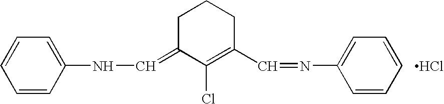 |
| 21 | ATT | O-(7-Azabenzotriazol-1-yl) N,N,N′,N′-tetramethyluronium hexafluorophosphate | 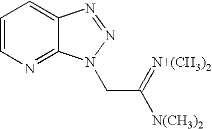 |
| 22 | AMF | 9-Aminofluorene hydrochloride | 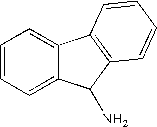 |
| 23 | BBL | Basic Blue | 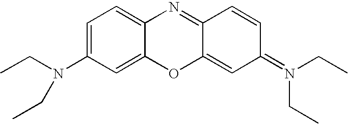 |
| 24 | DDA | 1,8-Diamino-4,5- dihydroxyanthraquinone | 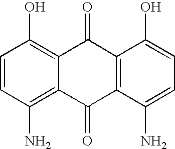 |
| 25 | PFV | Proflavine hemisulfate salt hydrate | 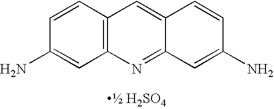 |
| 26 | APT | 2-Amino-1,1,3- propenetricarbonitrile | 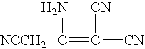 |
| 27 | VRA | Variamine Blue RT salt | 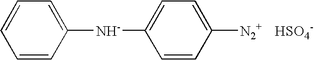 |
| 28 | TAP | 4,5,6-Triaminopyrimidine sulfate salt | 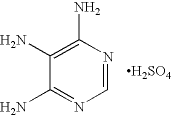 |
| 29 | ABZ | 2-Amino-benzothiazole | 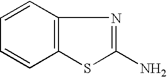 |
| 30 | MEL | Melamine | 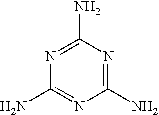 |
| 31 | PPN | 3-(3-Pyridylmethylamino) Propionitrile | 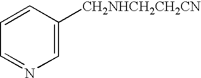 |
| 32 | SSD | Silver(I) Sulfadiazine | 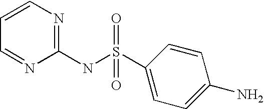 |
| 33 | AFL | Acriflavine | 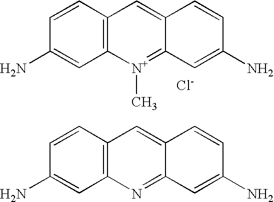 |
| 34 | AMPT | 4-Amino-6-mercaptopyrazolo[3,4- d]pyrimidine | 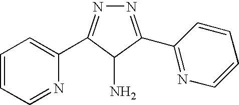 |
| 35 | APU | 2-Aminopurine | 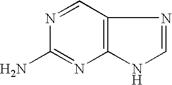 |
| 36 | ATH | Adenine Thiol | 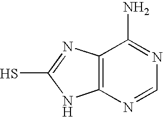 |
| 37 | FAD | Fluoroadenine | 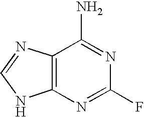 |
| 38 | MCP | 6-Mercaptopurine | 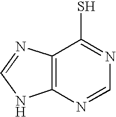 |
| 39 | AMP | 4-Amino-6-mercapyopyrazolo[3,4- d]pyrimidine | 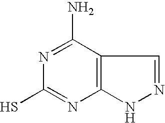 |
| 41 | R110 | Rhodamine 110 | 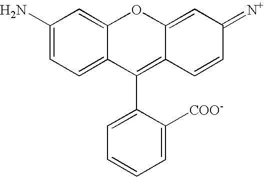 |
| 42 | ADN | Adenine | 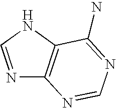 |
| 43 | AMB | 5-Amino-2-mercaptobenzimidazole | 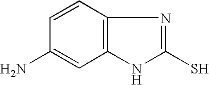 |

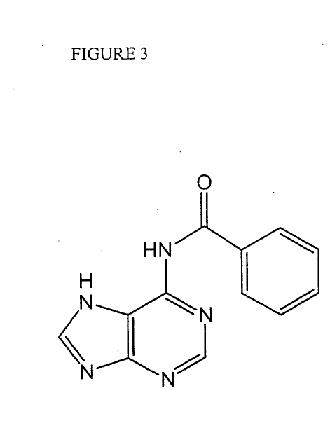

Many compounds that give strong regular Raman signals in solution do not yield strong signals in COINs. Further, within a particular compound, vibration modes that give strong regular Raman peaks in solution do not necessarily produce strong peaks in COINs. Strong signals from COINs are desirable in applications such as the detection of analytes that are present at low concentrations. Referring now to FIG. 3, a structure for an exemplary Raman label compound, N-benzoyl-adenine, is shown. In general, it has been discovered that compounds having the following attributes produce strong signals in COINs: (1) a conjugated aromatic system, preferably comprised of two or more rings; (2) at least one nitrogen or sulfur atom having a lone pair of electrons, and preferably at least two such atoms, and preferably the two such atoms are on the same geometric side of the molecule; (3) as few competing metal binding modes as possible. In FIG. 3, the exemplary Raman label compound contains two conjugated aromatic rings and a pair of nitrogen atoms on the same side of the molecule that are capable of chelating a metal (the 6-amino and 7-NH are capable of chelating a metal).

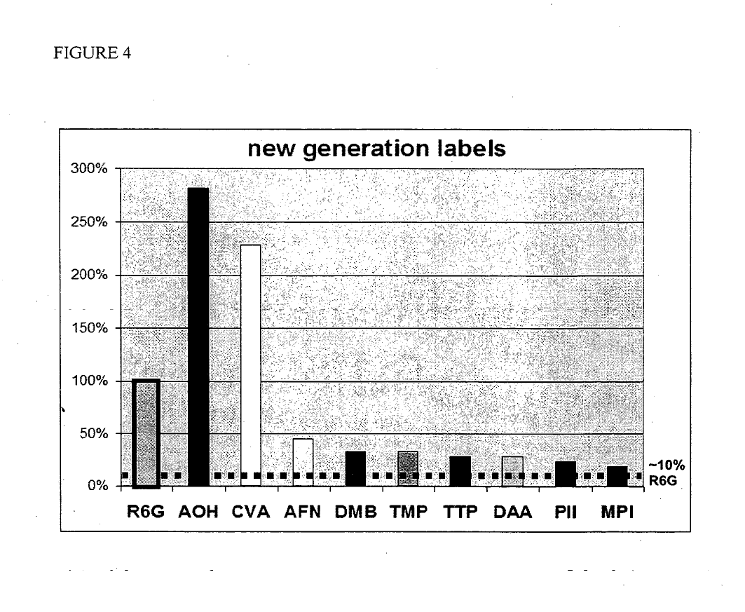

It was found that the organic compounds shown in Table 2 produced strong Raman signals upon incorporation into the COIN nanocluster. Referring now to FIG. 4, a comparison is provided between signal intensities achieved for COINs containing several different organic compounds. The vertical axis plots the label type and the abbreviations can be found in Table 2.

TABLE 2

| No. | Abbreviation | Name | Structure |
|---|---|---|---|
| 44 | AOH | Acridine Orange Hydrochloride | 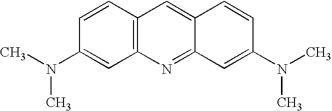 |
| 45 | CVA | Cresyl Violate Acetate | 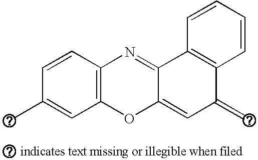 |
| 46 | AFN | Acriflavine Neutral | 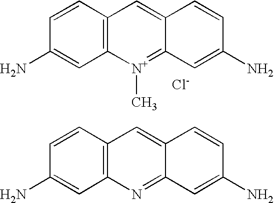 |
| 47 | DMB | Dimidium Bromide | 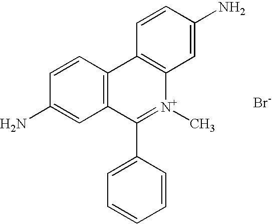 |
| 48 | TMP | 5,10,15,20-Tetrakis(N-methyl-4- pyridinio)porphyrin Tetra(p- toluenesulfonate) | 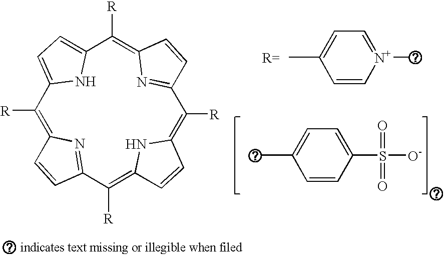 |
| 49 | TTP | 5,10,15,20-Tetrakis(4- trimethylaminophenyl)porphyrin Tetra(p-toluenesulfonate) | 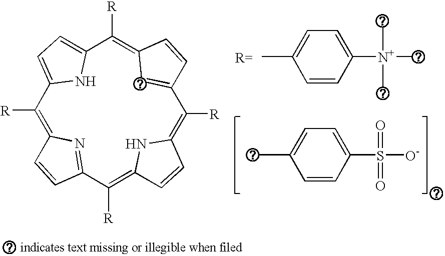 |
| 50 | DAA | 3,5-Diaminoacridine Hydrochloride | 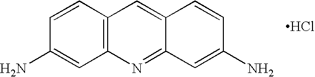 |
| 51 | PII | Propidium Iodide (3,8-diamino-5-(3- diethylaminopropyl)-6- phenylphenanthridinium iodide methiodide) | 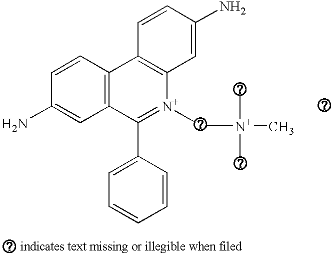 |
| 52 | MPI | Trans-4-[4-(dimethylamino)styryl]-1- methylpyridinium iodide | 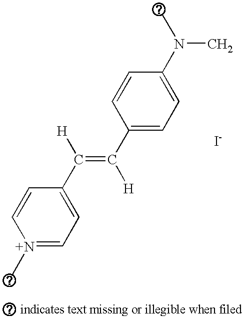 |
| 53 | DAB | 4-((4- (dimehtylamino)phenyl)azo)benzoic acid, succinimidyl ester | 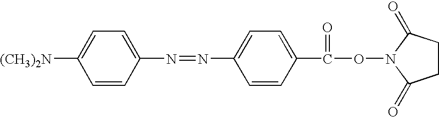 |

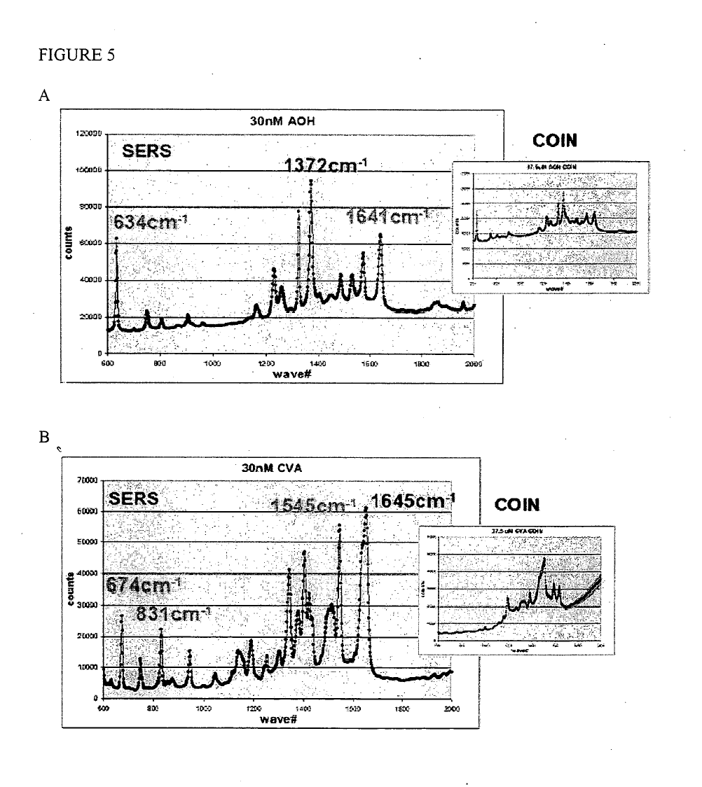

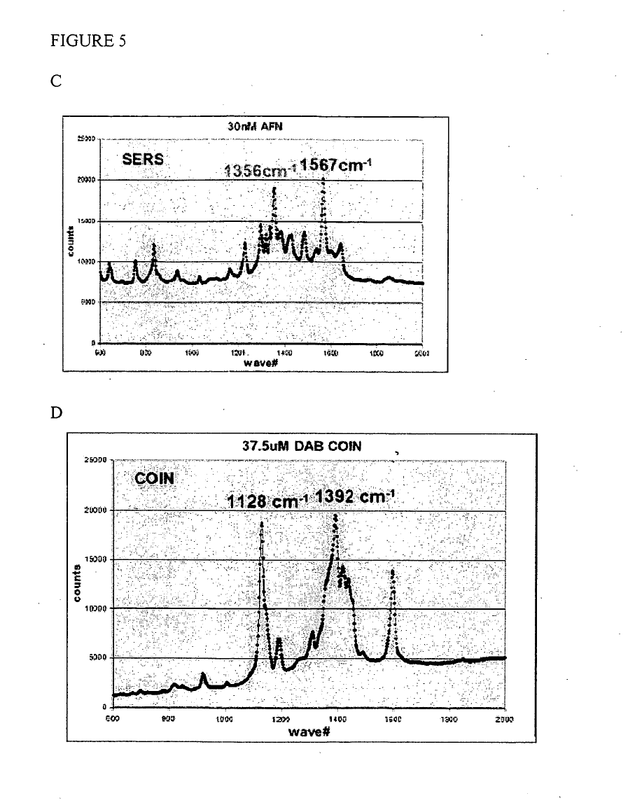

FIGS. 5A-D provide representative Raman spectra for COINs incorporating several of the organic Raman labels shown in Table 2. Spectra were obtained on a Mattec Renishaw Raman system.

In general, COINs can be prepared by causing colloidal metallic nanoparticles to aggregate in the presence of an organic Raman label. The colloidal metal nanoparticles can vary in size, but are chosen to be smaller than the desired size of the resulting COINs. For some applications, for example, in the oven and reflux synthesis methods, silver particles ranging in average diameter from about 3 to about 12 nm were used to form silver COINs and gold nanoparticles ranging from about 13 to about 15 nm were used to make gold COINs. In another application, for example, silver particles having a broad size distribution of about 10 to about 80 nm were used in a cold synthesis method. Additionally, multi-metal nanoparticles may be used, such as, for example, silver nanoparticles having gold cores.

To prepare colloidal metal nanoparticles, an aqueous solution is prepared containing suitable metal cations and a reducing agent. The components of the solution are then subject to conditions that reduce the metallic cations to form neutral, colloidal metal particles. Since the formation of the metallic clusters occurs in the presence of a suitable Raman-active organic compound, the Raman-active organic compound is readily incorporated onto the metal nanocluster during colloid formation. It is believed that the organic compounds trapped in the junctions between the primary metal particles provide the strongest Raman signal. A sample of COINs is typically comprised of COINs having COINs can typically be isolated by membrane filtration and COINS of different sizes can be enriched by centrifugation. Typical metals contemplated for use in formation of nanoclusters from metal colloids include, for example, silver, gold, copper, platinum, palladium, aluminum, gallium, indium, rhodium, and the like. In some embodiments the metal is silver or gold.

For organic Raman label compounds that tend to not cause colloid aggregation, an aggregation-inducing agent can be used. For example, the aggregation-inducing agent can be a salt, such as LiCl or NaCl, an acid or a base, such as HNO₃, HCl, or NaOH, or an organic compound, such as adenine, or benzyl-adenine. When aggregation-inducing agents are used, COIN synthesis can be performed at room temperature. Performing synthesis at room temperature is useful for making COINs from fluroescent dyes, since some of them can be unstable at elevated temperatures.

In general, for applications using COINs as reporters for analyte detection, the average diameter of the COIN particle should be less than about 200 nm. Typically, in analyte detection applications, COINs will range in average diameter from about 30 to about 200 nm. More preferably COINs range in average diameter from about 40 to about 200 nm, and more preferably from about 50 to about 200 nm, more preferably from about 50 to about 150 nm, and more preferably about 50 to about 100 nm. The thickness of the coating is, in one aspect, limited by the weight of the resulting particle and its ability to remain suspended in solution. For example, coatings that are lighter, such as protein coatings, can be thicker than heavier silica and metal coatings. Typically, coatings that are less than about 100 nm thick yield COINs that can be suspended in solution. Depending on the application desired, coatings can be as thin as about one layer of molecules.

Typical coatings useful in embodiments of the present invention include coatings such as metal layers, adsorption layers, silica layers, hematite layers, organic layers, and organic thiol-containing layers. Typically, the metal layer is different from the metal used to form the COIN. Additionally, a metal layer can typically be placed underneath any of the other types of layers. Many of the layers, such as the adsorption layers and the organic layers provide additional mechanisms for probe attachment. For instance, layers presenting carboxylic acid functional groups allow the covalent coupling of a biological probe, such as an antibody, through an amine group on the antibody.

Nanoclusters can include a second metal different from the first metal, wherein the second metal forms a layer overlying the surface of the nanocluster. Metals that can be used include for example, silver, gold, platinum, aluminum, copper, zinc, iron, and the like. In one example, the COIN is comprised of silver and the coating metal is gold. Typically, metal-coated COINs range in average diameter from about 20 to about 200 nm, from about 30 to about 200 nm, from about 40 to about 200 nm, from about 50 to about 200 nm, or more preferably from about 50 to about 150 nm. Typically, the thickness of the layer will depend on variables, such as, the size of the nanoparticle to which the coating is applied, the thickness of other layers to be added, changes to the plasma resonance wavelength induced by the thickness of the coating, the ability of the particles to remain suspended in the solution.

To prepare nanoparticles coated with a second metal, COINs are placed in an aqueous solution containing a suitable second metal (as a cation) and a reducing agent. The components of the solution are then subject to conditions that reduce the second metallic cations, thereby forming a metallic layer overlying the surface of the nanoparticle. Metal-coated COINs can be isolated and/or enriched in the same manner as uncoated COINs. In addition, COINs can be coated with a layer of gold by means of epitaxy growth. A procedure for growing gold particles developed by Zsigmondy and Thiessen (Das Kolloide Gold (Leipzig, 1925)), for example, may be employed. The growth medium contains chlorauric acid and hydroxylamine. The thickness of the gold coating can be controlled by the concentration of the COIN particles added to the growth medium.

COINs and metal-coated COINs can be functionalized through attachment of organic molecules to the surface. For example, gold and silver surfaces can be functionalized with a thiol-containing organic molecule to create an organic thiol layer. An organic molecule can be attached through well-known gold-thiol chemistry. The organic molecule can also contain a carboxyl group at the end distal from the thiol enabling further derivatization, such as attachment of a linker molecule, coating, a nucleic acid, or probe. In certain embodiments, the organic thiol-containing molecule is a branched or straight-chain carbon-containing molecule having 2 to about 20 carbon atoms. In additional embodiments, the organic thiol-containing molecule is a polymer, such as for example a polyethylene glycol, a polysaccharide, a peptide containing cysteine, or a mixture thereof. In further embodiments, the organic thiol-containing molecule is capable of binding to a single COIN via two or more thiol groups. In several non-limiting examples, the thiol can be, 2,3-disulfanyl-1-propanol, 3,4-disulfanyl-1-butanol, 4,5-disulfanyl-1-pentanol, 4-amino-2-thiomethyl-butanethiol, or 5-amino-2-thiomethyl-hexanethiol. In general, useful thiol-containing organic molecules have a molecular weight of less than about 9,000. However, in the case of soluble polymers having thiol groups, such as polycysteine, peptides containing cysteine, peptides containing homocysteine, polysaccharides containing thiol groups, or polyethylene glycol polymers containing thiol group(s), the molecular weight can be about 10,000 or less. The organic thiol-containing molecule may also contain one or more additional functional groups, such as groups that allow for coupling with a probe. Useful additional functional groups include, for example, carboxyl groups, esters, amines, photolabile groups, and alcohols. Additionally, suitable functional groups include, but are not limited to, hydrazide, amide, chloromethyl, epoxy, tosyl, and the like, which can be coupled to molecules such as probes through reactions commonly used in the art. Photolabile groups, by which is meant a functional group that can be activated by applying electromagnetic radiation (usually near IR, ultraviolet, or visible light) at a specific wavelength, include, for example, the types of groups disclosed in Aslam, M. and Dent, A., Bioconjugation: Protein Coupling Techniques for the Biomedical Sciences, Grove's Dictionaries, Inc., 301-316 (1998). These functional groups may also be further modified after attachment to a COIN to form more reactive species for coupling, such as for example, oxidizing an alcohol to an aldehyde. Useful thiol-containing molecules include, for example, sulfanylacetic acid, 3-sulfanylpropanoic acid, 6-sulfanylhexanoic acid, 5-sulfanylhexanoic acid, 4-sulfanylhexanoic acid, 3-sulfanylpropylacetate, 3-sulfanyl-1,2-propanediol (1-thioglycerol), 4-sulfanyl-2-butanol, 3-sulfanyl-1-propanol (3-mercapto-1-propanol), ethyl-3-sulfanylpropanoate, cysteine, homocysteine, 2-aminoethanethiol, 4-aminobutanethiol, 4-amino-2-ethyl-butanethiol, and other similar organic molecules having a molecular weight of about 9,000 or less. Additionally, the organic thiol layer may be composed of mixtures of different organic thiol-containing molecules. The mixtures of organic thiol-containing molecules may include thiols having an additional functional group, such as one for probe attachment, and thiols not having an additional functional group, as well as mixtures of thiols containing different functional groups or comprised of different organic molecules. In a further embodiment, the thiol-containing organic layer is attached to a COIN comprised of gold or silver or a COIN having a gold or a silver metal layer. Synthesis of organic thiol layers can be accomplished by standard techniques, such as placing the COINs to be coated in an aqueous solution containing the organic thiol.

In further embodiments of the present invention, COINs are coated with an adsorption layer. The adsorption layer can be comprised of, for example, an organic molecule or a polymer, such as for example, a block co-polymer or a biopolymer, such as for example, a protein, peptide, or a polysaccharide. The adsorption layer, in some cases, stabilizes the COINs and facilitates the reduction or prevention of further aggregation and precipitation from solution. This layer also can provide bio-compatible functional surfaces for probe attachment and aid in the prevention of non-specific binding to the COIN.

COINs can be coated with an adsorption layer comprised of an amphiphilic block copolymer. In particular, block copolymers having a hydrophobic region and a hydrophilic region can be adsorbed to the surface of a COIN via hydrophobic interactions. The hydrophilic region of the block copolymer aids in the dispersal of the COIN in aqueous media and can provide a site for coupling additional molecules, layers, or probes. The individual blocks that form a polymer molecule can be identical (homopolymer) or can be different (heteropolymer). Hydrophilic heteropolymers may comprise, for example, some blocks that are charged (for example, anionic) and some blocks that are uncharged. At a minimum, the copolymer should contain two different types of blocks, such as for example, AₓB_{y} or AₓB_{y}A_{z} where A and B represent the different hetero- or homopolymer units (in the case of homopolymers, these units are monomers) of a block copolymer and X, Y, and Z are natural numbers (a diblock copolymer), however polymers having additional different blocks are also useful, such as for example, AₓC_{y}B_{z} where A, B, and C represent different hetero- or homopolymer units (in the case of homopolymers these units are monomers) that make up a block copolymer and X, Y, and Z are natural numbers (a triblock copolymer). The number of repeating units that form each of the blocks of the block copolymer may the same or different. Additionally, the block copolymer may also contain functional groups for further modification or probe attachment. Typically, these functional groups will be located at or near the distal end of the hydrophilic section of the block copolymer for ease of further modification or probe attachment. Suitable functional groups include, but are not limited to carboxylic acid, esters, amines, hydroxyl, hydrazide, amide, chloromethyl, aldehyde, epoxy, tosyl, thiol, and the like, which can be coupled to molecules such as probes through reactions commonly used in the art. Further a coating may comprise a mixture of non-functionalized and functionalized copolymers, depending on the application and desire to adjust the number of probes or other molecules attached to a COIN surface. Ranges for mixtures of non-functionalized and functionalized block copolymers include, for example, about 10⁶:1 to about 1:10⁶ nonfunctionalized to functionalized block copolymer concentration. In general the block copolymer should have a molecular weight of about 1,000 to about 1,000,000, preferably about 1,000 to about 500,000, and more preferably about 1,000 to about 100,000. Suitable hydrophobic blocks include, but are not limited to, polyesters, polystyrenes, polyethylacrylate, polybutylacrylate, poly(propylene oxide), and poly(ethylene oxide). Suitable hydrophilic blocks include, but are not limited to, polyacrylamide/polyacrylic acid copolymers, poly(L-amino acid)s, poly(2-methacryloxyethyltrimethyl ammonium bromide), polystyrenesulfonic acid, and polystyrene-polystyrenesulfonic acid copolymers. For example the block copolymer can be a poly(L-amino acid)-block-polyester-block, polyglycol-block-poly(L-amino acid)-block, or a polystyrene-block-polystyrenesulfonic acid-block. Additional examples include, but are not limited to, polystyrene-block-poly(4-vinylpyridine)-block; polystyrene-block-poly(2-vinylpyridine)-block; polystyrene-block-poly(4-vinylphenol)-block; poly(4-vinylpyridine-block-poly(butyl methacrylate)-block; polystyrene-block-poly(maleic acid)-block; and poly(viny-1-chloride-co-vinyl acetate)-block-poly(maleic acid)-block. A block copolymer layer can be adsorbed on a COIN or a COIN coated with a metal layer, such as, for example, a silver COIN coated with a gold protection layer. A block copolymer adsorption layer can be prepared by dissolving the block copolymer in an aqueous solution containing the COINs and allowing the block copolymer to associate with the surface of the COINs.

In a further embodiment, the COIN or the COIN having a metal layer is coated with an adsorbed layer of protein. Suitable proteins include non-enzymatic soluble globular or fibrous proteins. For applications involving molecular detection, the protein should be chosen so that it does not interfere with a detection assay, in other words, the proteins should not also function as competing or interfering probes in a user-defined assay. By non-enzymatic proteins is meant molecules that do not ordinarily function as biological catalysts. Examples of suitable proteins include avidin, streptavidin, bovine serum albumen (BSA), transferrin, insulin, soybean protein, casine, gelatine, and the like, and mixtures thereof. A bovine serum albumen layer affords several potential functional groups, such as, carboxylic acids, amines, and thiols, for further functionalization or probe attachment. Optionally, the protein layer can be cross-linked with EDC, or with glutaraldehyde followed by reduction with sodium borohydride.

In additional embodiments a COIN or a metal-coated COIN is coated with a soluble polymeric adsorption layer. In general the soluble polymer should have a molecular weight of about 1,000 to about 1,000,000, preferably about 1,000 to about 500,000, and more preferably about 1,000 to about 100,000. For example, suitable polymeric adsorption layers include, polyacrylamide, partially hydrolyzed polyacrylamide, polyacrylic acid, polyacrylamide acrylic acid copolymers, polybutadiene-maleic acid copolymers, polyglycol-poly(L-amino acid) copolymers, polyethylenimine (branched or unbranched), PEG-PE (polyethylene glycol-phosphoethanolamine), poly(L-lysine hydrobromide), PGUA (polygalacturonic acid), or algenic acid. A polyacrylic acid layer adsorbed onto a COIN provides a carboxylic acid functional group for further derivatization or probe attachment though, for example, EDC coupling. A polymer adsorption layer can be prepared by dissolving the polymer in an aqueous solution containing COINs and allowing the polymer to associate with the surface of the COINs.

As an alternative to metallic protection layers or in addition to metallic protection layers, COINs can be coated with a layer of silica. Silica deposition is initiated from a supersaturated silica solution, followed by growth of a silica layer through ammonia-catalyzed hydrolysis of tetraethyl orthosilicate (TEOS). COINs can be coated with silica and functionalized, for example, with an organic amine-containing group. A silver COIN or silver- or gold-coated COIN can be coated with a layer of silica via the procedure described in V. V. Hardikar and E. Matijevic, J. Colloid Interface Science 221:133-136 (2000). Additionally, silica-coated COINs are readily functionalized using standard silica chemistry. For example, a silica-coated COIN can be derivatized with (3-aminopropyl)triethoxysilane to yield a silica coated COIN that presents an amine group for further coating, layering, modification, or probe attachment. See, for example, Wong, C., Burgess, J., Ostafin, A., “Modifying the Surface Chemistry of Silica Nano-Shells for Immunoassays,” Journal of Young Investigators, 6:1 (2002), and Ye, Z., Tan, M., Wang, G., Yuan, J., “Preparation, Characterization, and Time-Resolved Fluorometric Application of Silica-Coated Terbium(III) Fluorescent Nanoparticles,” Anal. Chem., 76:513 (2004). Additional layers or coatings that may be layered on a silica coating include the coatings and layers exemplified herein.

COINs can also include an organic layer. This organic layer can overlie another layer, such as a metal layer or a silica layer. An organic layer can also be used to provide colloidal stability and functional groups for further modification. The organic layer is optionally crosslinked to form a more unified coating. An exemplary organic layer is produced by adsorption of an octylamine modified polyacrylic acid onto COINs, the adsorption being facilitated by the positively charged amine groups. The carboxyl groups of the polymer are then crosslinked with a suitable agent such as lysine, (1,6)-diaminoheptane, or the like. Unreacted carboxyl groups can be used for further derivation or probe attachment, such as through EDC coupling.

The COINs of the present invention can perform as sensitive reporters for use in fluid-based molecular analyte detection, and also for highly parallel analyte detection. A set of COINs can be created in which each member of the set has a Raman signature unique to the set. Any of the types of COINs as discussed above can be used for analyte detection. In general, COINs-can range in average diameter from about 20 nm to about 200 nm, and for analyte detection, preferably from about 30 to about 200 nm, from about 40 to about 200 nm, or more preferably from 50 to about 150 nm.

COINs can be complexed to the molecular analyte through a probe attached to the COIN. In general, a probe is a molecule that is able to specifically bind an analyte and, in certain embodiments, exemplary probes are antibodies, antigens, polynucleotides, oligonucleotides, carbohydrates, proteins, cofactors, receptors, ligands, peptides, inhibitors, activators, hormones, cytokines, and the like. For example, the analyte can be a protein and the COIN is complexed to the analyte through an antibody that specifically recognizes the protein analyte of interest.

In some embodiments, a probe is an antibody. As used herein, the term antibody is used in its broadest sense to include polyclonal and monoclonal antibodies, as well as antigen binding fragments of such antibodies. An antibody useful the present invention, or an antigen binding fragment thereof, is characterized, for example, by having specific binding activity for an epitope of an analyte. An antibody, for example, includes naturally occurring antibodies as well as non-naturally occurring antibodies, including, for example, single chain antibodies, chimeric, (DR-grafted, bifunctional, and humanized antibodies, as well as antigen-binding fragments thereof. Such non-naturally occurring antibodies can be constructed using solid phase peptide synthesis, can be produced recombinantly, or can be obtained, for example, by screening combinatorial libraries consisting of variable heavy chains and variable light chains.

Additionally, a probe can be a polynucleotide. A COIN-labeled oligonucleotide probe can be used in a hybridization reaction to detect a target polynucleotide. Polynucleotide is used broadly herein to mean a sequence of deoxyribonucleotides or ribonucleotides that are linked together by a phosphodiester bond. Generally, an oligonucleotide useful as a probe or primer that selectively hybridizes to a selected nucleotide sequence is at least about 10 nucleotides in length, usually at least about 15 nucleotides in length, for example between about 15 and about 50 nucleotides in length. Polynucleotide probes are particularly useful for detecting complementary polynucleotides in a biological sample and can also be used for DNA sequencing by pairing a known polynucleotide probe with a known Raman-active signal made up of a combination of Raman-active organic compounds as described herein.

A polynucleotide can be RNA or DNA, and can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (for example, a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. One example of an oligomeric compound or an oligonucleotide mimetic that has been shown to have good hybridization properties is referred to as a peptide nucleic acid (PNA). In PNA compounds, the sugar-backbone of an oligonucleotide is replaced with an amide containing backbone, for example an aminoethylglycine backbone. In this example, the nucleobases are retained and bound directly or indirectly to an aza nitrogen atom of the amide portion of the backbone. PNA compounds are disclosed in Nielsen et al., Science, 254:1497-15 (1991), for example.

The covalent bond linking the nucleotides of a polynucleotide generally is a phosphodiester bond. However, the covalent bond also can be any of a number of other types of bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs can be particularly useful where the polynucleotide is to be exposed to an environment that can contain nucleolytic activity, including, for example, a tissue culture medium or upon administration to a living subject, since the modified polynucleotides can be less susceptible to degradation.

An analyte can be any molecule or compound in the solid, liquid, gaseous or vapor phase. By gaseous or vapor phase analyte is meant a molecule or compound that is present, for example, in the headspace of a liquid, in ambient air, in a breath sample, in a gas, or as a contaminant in any of the foregoing. It will be recognized that the physical state of the gas or vapor phase can be changed for example, by pressure, temperature as well as by affecting surface tension of a liquid by the presence of or addition of salts.

The analyte can be comprised of a member of a specific binding pair (sbp) and may be a monovalent ligand (monoepitopic) or polyvalent ligand (polyepitopic), usually antigenic or haptenic, and is a single compound or plurality of compounds which share at least one common epitopic or determinant site. The analyte can be derived from a cell such as bacteria or a cell bearing a blood group antigen such as A, B, D, etc., or an HLA antigen or a microorganism, for example, bacterium, fungus, protozoan, prion, or virus. In certain aspects of the invention, the analyte is charged. A biological analyte could be, for example, a protein, a carbohydrate, or a nucleic acid.

The nanoparticles of the present invention may be used to detect the presence of a particular target analyte, for example, a protein, enzyme, polynucleotide, carbohydrate, antibody, or antigen. The nanoparticles may also be used to screen bioactive agents, such as, drug candidates, for binding to a particular target or to detect agents like pollutants. As discussed above, any analyte for which a probe moiety, such as a peptide, protein, or aptamer, may be designed can be used in combination with the disclosed nanoparticles.

Molecular analytes include antibodies, antigens, polynucleotides, oligonucleotides, proteins, enzymes, polypeptides, polysaccharides, cofactors, receptors, ligands, and the like. The analyte may be a molecule found directly in a sample such as a body fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like. Methods for detecting target nucleic acids are useful for detection of infectious agents within a clinical sample, detection of an amplification product derived from genomic DNA or RNA or message RNA, or detection of a gene (cDNA) insert within a clone. Detection of the specific Raman label on the captured COIN labeled oligonucleotide probe identifies the nucleotide sequence of the oligonucleotide probe, which in turn provides information regarding the nucleotide sequence of the target polynucleotide.

In addition, the detection target can be any type of animal or plant cell, or unicellular organism. For example, an animal cell could be a mammalian cell such as an immune cell, a cancer cell, a cell bearing a blood group antigen such as A, B, D, etc., or an HLA antigen, or virus-infected cell. Further, the target cell could be a microorganism, for example, bacterium, algae, or protozoan. The molecule bound by the probe is present on the surface of the cell and the cell is detected by the presence of a known surface feature (analyte) through the complexation of a COIN to the target cell-surface feature. In general, cells can be analyzed for one or more surface features through the complexation of at least one uniquely labeled COIN to a known surface feature of a target cell. Additional surface features can be detected through the complexation of a differently labeled COIN to a second known surface feature of the target cell, or the complexation of two differently labeled COINs to a second and third surface feature, and so on. One or more cells can be analyzed for the presence of a surface feature through the complexation of a uniquely labeled COIN to a known surface feature of a target cell.

Cell surface targets include molecules that are found attached to or protruding from the surface of a cell, such as, proteins, including receptors, antibodies, and glycoproteins, lechtins, antigens, peptides, fatty acids, and carbohydrates. The cellular analyte may be found, for example, directly in a sample such as fluid from a target organism. The sample can be examined directly or may be pretreated to render the analyte more readily detectible. The fluid can be, for example, urine, blood, plasma, serum, saliva, semen, stool, sputum, cerebral spinal fluid, tears, mucus, and the like. The sample could also be, for example, tissue from a target organism.

In general, probes can be attached to metal-coated COINs through adsorption of the probe to the COIN surface. Alternatively, COINs may be coupled with probes through biotin-avidin coupling. For example, avidin or streptavidin (or an analog thereof) can be adsorbed to the surface of the COIN and a biotin-modified probe contacted with the avidin or streptavidin-modified surface forming a biotin-avidin (or biotin-streptavidin) linkage. As discussed above, optionally, avidin or streptavidin may be adsorbed in combination with another protein, such as BSA, and/or optionally crosslinked. In addition, for COINs having a functional layer that includes a carboxylic acid or amine functional group, probes having a corresponding amine or carboxylic acid functional group can be attached through water-soluble carbodiimide coupling reagents, such as EDC (1-ethyl-3-(3-dimethyl aminopropyl)carbodiimide), which couples carboxylic acid functional groups with amine groups. Further, functional layers and probes can be provided that possess reactive groups such as, esters, hydroxyl, hydrazide, amide, chloromethyl, aldehyde, epoxy, tosyl, thiol, and the like, which can be joined through the use of coupling reactions commonly used in the art. For example, Aslam, M and Dent, A, Bioconjugation: Protein Coupling Techniques for the Biomedical Sciences, Grove's Dictionaries, Inc., (1998) provides additional methods for coupling biomolecules, such as, for example, thiol maleimide coupling reactions, amine carboxylic acid coupling reactions, amine aldehyde coupling reactions, biotin avidin (and derivatives) coupling reactions, and coupling reactions involving amines and photoactivatable heterobifunctional reagents.

Nucleotides attached to a variety of tags may be commercially obtained (for example, from Molecular Probes, Eugene, Oreg.; Quiagen (Operon), Valencia, Calif.; and IDT (Integrated DNA Technologies), Coralville, Iowa) and incorporated into oligonucleotides or polynucleotides. Oligonucleotides may be prepared using commercially available oligonucleotide synthesizers (for example, Applied Biosystems, Foster City, Calif.). Additionally, modified nucleotides may be synthesized using known reactions, such as for example, those disclosed in, Nelson, P., Sherman-Gold, R., and Leon, R., “A New and Versatile Reagent for Incorporating Multiple Primary Aliphatic Amines into Synthetic Oligonucleotides,” Nucleic Acids Res., 17:7179-7186 (1989) and Connolly, B., Rider, P., “Chemical Synthesis of Oligonucleotides Containing a Free Sulfhydryl Group and Subsequent Attachment of Thiol Specific Probes,” Nucleic Acids Res., 13:4485-4502 (1985). Alternatively, nucleotide precursors may be purchased containing various reactive groups, such as biotin, hydroxyl, sulfhydryl, amino, or carboxyl groups. After oligonucleotide synthesis, COIN labels may be attached using standard chemistries. Oligonucleotides of any desired sequence, with or without reactive groups for COIN attachment, may also be purchased from a wide variety of sources (for example, Midland Certified Reagents, Midland, Tex.).

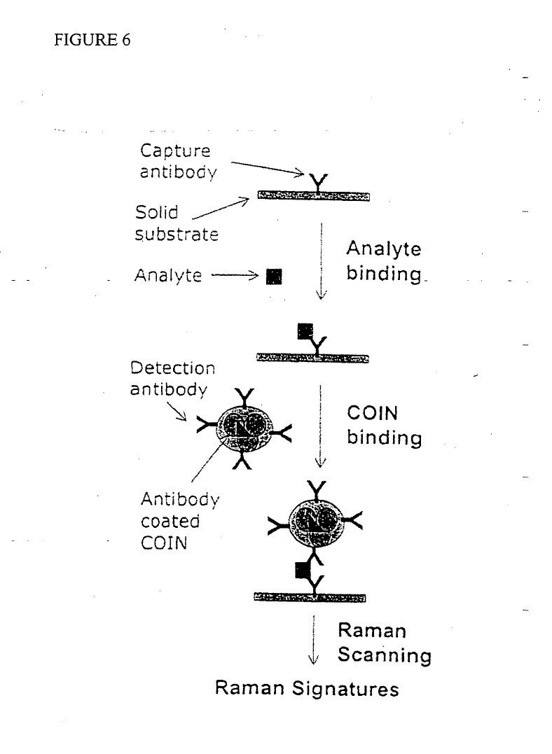

Referring now to FIG. 6, FIG. 6 diagrams a method that uses COINs as reporters for analyte detection. In this example a solution containing a known analyte is contacted with a substrate surface under conditions that allow the known analyte to be immobilized on the substrate surface through binding to a capture antibody specific for the analyte (an antibody that recognizes a first epitope of the analyte), attached to the substrate surface. A COIN having an attached probe specific for the analyte, in this case an antibody that recognizes a second epitope of the analyte, is then contacted with the substrate surface under conditions that allow the COIN-antibody conjugate to bind to the analyte. Unbound COINs are then washed from the substrate surface. The detection of the Raman signature of a COIN on the substrate surface indicates the presence of the analyte in the analysis sample. This analysis can also be performed in a multiplexed fashion. A set of COINs can be created having unique signatures and probes specific for two or more known analytes in a sample. Similarly, arrays having multiple desired probes for analytes can be created. In this case the detection of each unique COIN signal is indicative of the presence of a specific known analyte in the analysis sample.

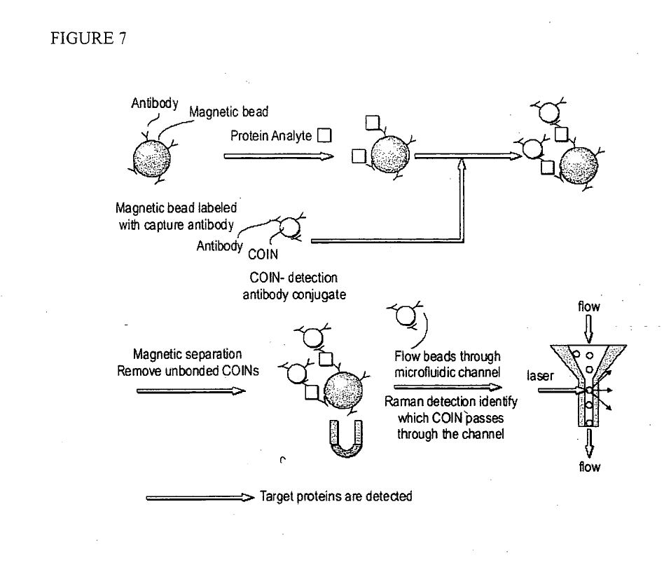

Microspheres having an attached probe can be contacted with the analyte solution and used to separate target analytes from uncomplexed COINs. The microsphere carriers are complexed to the analytes of interest via the types of probes and specific binding interactions discussed above for the complexation of COINs to analytes. For example, the complexation of a microsphere to a target analyte can occur through antibodies, receptors, inhibitors, activators, hormones, or nucleic acid probes. Thus, if antibodies are used, the microsphere is conjugated to one or more antibodies that recognize a first epitope on the target molecule, and the COIN is conjugated to one or more antibodies that recognize a second epitope on the same target molecule. In an alternate example, the COIN is conjugated to a ligand and the microsphere is conjugated to an antibody that recognizes the receptor for the ligand, or vice versa. If the target analyte is a polynucleotide, the COIN is conjugated to an oligonucleotide probe complementary to a section of the polynucleotide and the microsphere is conjugated to an oligonucleotide probe that recognizes a different section of the target polynucleotide. The microsphere carriers can be, for example, latex, polystyrene, agarose, or surface-coated magnetic beads. The microspheres typically are about 0.1 to about 50 μm, preferably about 0.5 to about 25 μm, and more preferably about 1 to about 10 μm in diameter. Useful microspheres are available from, for example, Polysciences, Warrington, Pa.; Dynal Biotech Inc., Brown Deer, Wis.; Magsphere, Inc., Pasadena, Calif.; and Bangs Laboratories, Inc., Fishers, Ind. In general, microspheres that allow for size-based separation of the microspheres from the uncomplexed COINs are useful. Optionally, the microsphere carriers may contain a Raman label, such as COINs, or a fluorescent label. These microsphere-analyte-COIN complexes can be separated from uncomplexed COINs using the flow characteristics of the microspheres or centrifugation. Thus, an analyte, complexed with a microsphere that is larger than the COINs used in the method, could be separated from unbound COINs in a fluid flow through a channel or microchannel because the larger microspheres move more slowly through the channel. Alternately, the microsphere carriers can be magnetic microspheres which can be separated from the reaction mixture by magnetic force. In this embodiment, free COINs are washed away and COINs complexed with the analyte and magnetic microsphere are left (FIG. 7). The complexes are then resuspended by removal of the magnetic field. Alternatively, the carrier microsphere beads can be separated from unbound COINs using affinity binding. In this embodiment, the microsphere bead contains an affinity ligand, such as biotin, that can be captured by a specific receptor, such as avidin. The complexed analyte is then separated from uncomplexed COINs through affinity attachment to a solid support and washing away of the uncomplexed COINs. Other types of affinity attachment ligands include lectin-sugar interactions, phage-displayed antibodies, or single chain antibodies with antigens. The complexes are then resuspended (for example, in 1× PBS buffer). The purified microsphere-analyte-COIN complexes are passed through a detection channel operably coupled with a Raman spectrometer. Optionally, the COINs can be separated from the analyte complex before detection. COINs can be separated from the complex using conditions such as high (greater than about 10) or low (less than about 4) pH, low salt concentration (less than about 1 mM), protease digestion, or using protein denaturing conditions such as heating (greater than about 50° C.) and high surfactant concentration (for example, greater than about 1% Tween™-20 or SDS), depending on the method of probe attachment. For example, if the probe is an antibody or other protein the forgoing conditions can be used to digest the complex, if the probe is a nucleic acid, conditions such a low salt solutions (less than about 1 mM salt), heating to above the melting temperature of the probe-complementary strand complex, nuclease digestion, and binding replacement (by PNA, for example).

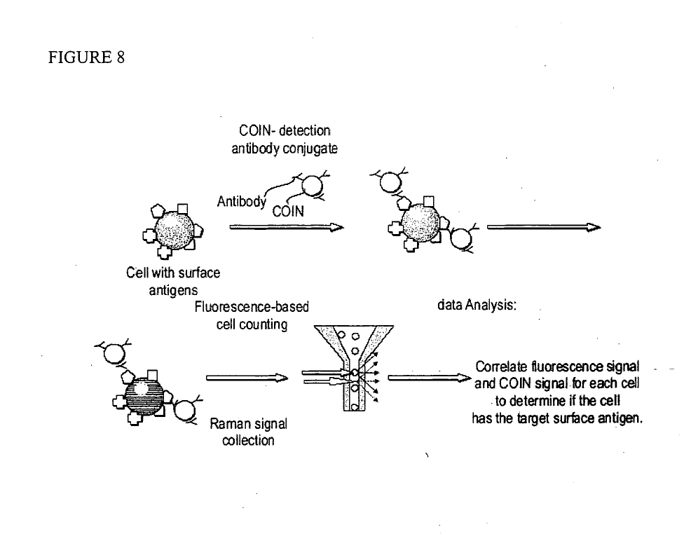

In an embodiment of the present invention, a cellular analyte solution is contacted with a COIN having a probe specific for a known cell surface antigen. For example, in FIG. 8, a cell is contacted with a COIN having attached antibody probes specific for a surface antigen. The COIN is complexed to the cell through the specific binding of the probe to a cell surface analyte. The cell is optionally fluorescently stained. Typical fluorescent dyes that can be used for cellular staining include 1,4-diacetoxy-2,3-dicyano-benzene (ADB) (available from Sigma Chemicals, St. Louis, Mo.), 3,3-dihexyl-oxacarbocyanin (available from Eastman Kodak, Rochester, N.Y.), rhodamine 123 (available from Sigma Chemicals, St. Louis, Mo.), 2′,7′-dichlorofluorescin-diacetate (available from Sigma Chemicals, St. Louis Mo.), 2′,7′-dichlorofluorescein (available from Sigma Chemicals, St. Louis, Mo.), FLUO-3 AM cell permeant (available from Molecular Probes Inc., Eugene, Oreg.), acridine orange (available from Polysciences, Warrington, Pa.), propidium iodide (available from Sigma, St. Louis, Mo.), and hydroethidine (available from Polysciences, Warrington, Pa.). The cellular analytes are then separated from uncomplexed COINs (this can be accomplished in a fluid flow that allows the smaller uncomplexed COINs to travel faster with the flow than the larger cells, or through centrifugation that fractionates larger heavier complexed cells from uncomplexed COINs, for example) and passed through a detector cavity where the fluorescence from the dye and the Raman signal from the COIN are collected. Correlation of the COIN Raman signature with the fluorescent signal indicates that the cell surface is presenting the target antigen. Additionally, detection of fluorescent signal provides information regarding the total number of cellular analytes present in the sample. Alternately, the cell may be complexed with a second COIN having a Raman label that is different from the first. This Raman label may be complexed using a probe that is specific for the same or for a different cell surface feature as that recognized by the probe associated with the first COIN. The cellular analytes are then separated from uncomplexed COINs (this can be accomplished in a fluid flow that allows the smaller uncomplexed COINs to travel faster with the flow than the larger cells, through centrifugation that fractionates larger heavier complexed cells from uncomplexed COINs, or by dilution, for example) and passed through a detector cavity where the signals from the COINS are collected. Co-detection of two different COIN signatures indicates the presence of the target cell. If the unique COINs are associated with probes that are specific for different cell surface features, the co-occurrence of the two COIN signatures also indicates the presence of two different features on the cell surface. Optionally, the cells are also fluorescently stained. The detection of a fluorescence signal confirms the presence of cells and allows information to be acquired regarding the total number of cells present in the sample.

In the practice of embodiments of the present invention, a Raman spectrometer can be part of a detection unit designed to detect and quantify nanoparticles of the present invention by Raman spectroscopy. Methods for detection of Raman labeled analytes, for example nucleotides, using Raman spectroscopy are known in the art. (See, for example, U.S. Pat. Nos. 5,306,403; 6,002,471; 6,174,677). A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled nanoparticles is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, which includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternate excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6X). The objective lens may be used to both excite the Raman-active organic compounds of the COINs and to collect the Raman signal, by using a holographic beam splitter (Kaiser Optical Systems, Inc., Model HB 647-26N 18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enchanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection of the nanoparticles of the present invention, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

Raman signatures from COINs can be analyzed, for example, using data signature and peak analysis through peak fitting. Scanned data were analyzed for signature profiles such as Raman peak intensities and locations as well as peak width. The data were analyzed using peak and curve fitting algorithms to identify statistically the most likely parameters (such as for example, wave number, intensity, peak width, and associated baseline values) that come from control experiments, such as for example, signals from water, solvent, the substrate, and or system noise. Raman peak intensities were normalized to methanol's first main peak and, where required, also further normalized to label concentrations.

### EXAMPLE 1

Synthesis

Chemical reagents: Biological reagents including anti-IL-2 and anti-IL-8 antibodies were purchased from BD Biosciences Inc. The capture antibodies were monoclonal antibodies generated from mouse. Detection antibodies were polyclonal antibodies generated from mouse and conjugated with biotin. Aqueous salt solutions and buffers were purchased from Ambion, Inc. (Austin, Tex., USA), including 5 M NaCl, 10× PBS (1× PBS 137 mM NaCl, 2.7 mM KCl, 8 mM Na₂HPO₄, and 2 mM KH₂PO₄, pH 7.4). Unless otherwise indicated, all other chemicals were purchased, at highest available quality, from Sigma Aldrich Chemical Co. (St. Louis, Mo., USA). Deionized water used for experiments had a resistance of 18.2×106 Ohms-cm and was obtained with a water purification unit (Nanopure Infinity, Barnstad, USA).

Silver seed particle synthesis: Stock solutions (0.500 M) of silver nitrate (AgNO₃) and sodium citrate (Na₃Citrate) were filtered twice through 0.2 micron polyamide membrane filters (Schleicher and Schuell, NH, USA) which were thoroughly rinsed before use. Sodium borohydrate solution (50 mM) was made fresh and used within 2 hours. Silver seed particles were prepared by rapid addition of 50 mL of Solution A (containing 8.00 mM Na₃Citrate, 0.60 mM sodium borohydrate, and 2.00 mM sodium hydroxide) into 50 mL of Solution B (containing 4.00 mM silver nitrate) under vigorous stirring. Addition of Solution B into Solution A led to a more polydispersed suspension. Silver seed suspensions were stored in the dark and used within one week. Before use, the suspension was analyzed by Photon Correlation Spectroscopy (PCS, Zetasizer 3000 HS or Nano-ZS, Malvern) to ensure the intensity-averaged diameter (z-average) was between 10-12 nm with a polydispersity index of less than 0.25.

Gold seed particle synthesis: A household microwave oven (1350W, Panasonic) was used to prepare gold nanoparticles. Typically, 40 mL of an aqueous solution containing 0.500 mM HAuCl₄ and 2.0 mM sodium citrate in a glass bottle (100 mL) was heated to boiling in the microwave using the maximum power, followed by a lower power setting to keep the solution gently boiling for 5 min. 2.0 grams of PTFE boiling stones (6 mm, Saint-Gobain A1069103, through VWR) were added to the solution to promote gentle and efficient boiling. The resultant solutions had a rosy red color. Measurements by PCS showed that the gold solutions had a typical z-average of 13 nm with a polydispersity index of less than 0.04.

COIN Synthesis: In general, Raman labels were pipetted into the COIN synthesis solution to yield final concentrations of the labels in synthesis solution of about 1 to about 50 μM. Stock solutions of Raman labels were prepared having concentraions of about 0.25 mM to about 1 mM of ultra-purified water. In some cases, acid or organic solvents, such as, for example, ethanol, were used to enhance label solubility. For example, 8-aza-adenine and N-benzoyladenine were pipetted into the COIN formation reaction as 1.00 mM solutions in 1 mM HCl, 2-mercapto-benzimidazole was added from a 1.0 mM solution in ethanol, and 4-amino-pyrazolo[3,4-d]pyrimidine and zeatin were added from a 0.25 mM solution in 1 mM HNO₃.

Reflux method: To prepare COIN particles with silver seeds, typically, 50 mL silver seed suspension (equivalent to 2.0 mM Ag⁺) was heated to boiling in a reflux system before introducing Raman labels. Silver nitrate stock solution (0.50 M) was then added dropwise or in small aliquots (50-100 μL) to induce the growth and aggregation of silver seed particles. Up to a total of 2.5 mM silver nitrate could be added. The solution was kept boiling until the suspension became very turbid and dark brown in color. At this point, the temperature was lowered quickly by transferring the colloid solution into a glass bottle. The solution was then stored at room temperature. The optimum heating time depended on the nature of Raman labels and amounts of silver nitrate added. It was found helpful to verify that particles had reached a desired size range (80-100 nm on average) by PCS or UV-Vis spectroscopy before the heating was arrested. Normally, a dark brown color was an indication of cluster formation and associated Raman activity.

To prepare COIN particles with gold seeds, typically, gold seeds were first prepared from 0.25 mM HAuCl₄ in the presence of a Raman label (for example, 20 μM 8-aza-adenine). After heating the gold seed solution to boiling, silver nitrate and sodium citrate stock solutions (0.50 M) were added, separately, so that the final gold suspension contained 1.0 mM AgNO₃ and 1.0 mM sodium citrate. Silver chloride precipitate might form immediately after silver nitrate addition but disappeared soon with heating. After boiling, an orange-brown color developed and stabilized. An additional aliquot (50-100 μL) of silver nitrate and sodium citrate stock solutions (0.50 M each) was added to induce the development of a green color, which was the indication of cluster formation and was associated with Raman activity.

Note that the two procedures produced COINs with different colors, primarily due to differences in the size of primary particles before cluster formation.

Oven method: COINs can also be prepared conveniently by using a convection oven. Silver seed suspension was mixed with sodium citrate and silver nitrate solutions in a 20 mL glass vial. The final volume of the mixture was typically 10 mL, which contained silver particles (equivalent to 0.5 mM Ag⁺), 1.0 mM silver nitrate and 2.0 mM sodium citrate (including the portion from the seed suspension). The glass vials were incubated in the oven, set at 95 ° C, for 60 min. before being stored at room temperature. A range of label concentrations could be tested at the same time. Batches showing brownish color with turbidity were tested for Raman activity and colloidal stability. Batches with significant sedimentation (which occurred when the label concentrations were too high) were discarded. Occasionally, batches that did not show sufficient turbidity could be kept at room temperature for an extended period of time (up to 3 days) to allow cluster formation. In many cases, suspensions became more turbid over time due to aggregation, and strong Raman activity developed within 24 hours. A stabilizing agent, such as bovine serum albumin (BSA), could be used to stop the aggregation and stabilize the COIN suspension.

A similar approach was used to prepare COINs with gold cores. Briefly, 3 mL of gold suspensions (0.50 mM Au³⁺) prepared in the presence of Raman labels was mixed with 7 mL of silver citrate solution (containing 5.0 mM silver nitrate and 5.0 mM sodium citrate before mixing) in a 20 mL glass vial. The vial was placed in a convection oven and heated to 95 ° C. for 1 hour. Different concentrations of labeled-gold seeds could be used simultaneously in order to produce batches with sufficient Raman activities.

Cold Method: 100 mL of silver particles (1 mM silver atoms) were mixed with 1 mL of Raman label solution (typically 1 mM). Then, 5 to 10 mL of 0.5 M LiCl solution was added to induce silver aggregation. As soon as the suspension became visibly darker (due to aggregation), 0.5% BSA was added to inhibit the aggregation process. Afterwards, the suspension was centrifuged at 4500 g for 15 minutes. After removing the supernatant (mostly single particles), the pellet was resuspended in 1 mM sodium citrate solution. The washing procedure was repeated for a total of three times. After the last washing, the resuspended pellets were filtered through 0.2 μM membrane filter to remove large aggregates. The filtrate was collected as COIN suspension. The concentrations of COINs were adjusted to 1.0 or 1.5 mM with 1 mM sodium citrate by comparing the absorbance at 400nm with 1 mM silver colloids for SERS.

It should be noted that a COIN sample can be heterogeneous in terms of size and Raman activity. We typically used centrifugation (200-2,000×g for 5-10 min.) or filtration (300 kDa, 1000 kDa, or 0.2 micron filters, Pall Life Sciences through VWR) to enrich for particles in the range of 50-100 nm. It is recommended to coat the COIN particles with a protection agent (for example, BSA, antibody) before enrichment. Some lots of COINs that we prepared (with no further treatment after synthesis) were stable for more than 3 months at room temperature without noticeable changes in physical and chemical properties.

Particle size measurement: The sizes of silver and gold seed particles as well as COINs were determined by using Photon Correlation Spectroscopy (PCS, Zetasizer3 3000 HS or Nano-ZS, Malvern). All measurements were conducted at 25 ° C. using a He—Ne laser at 633 nm. Samples were diluted with DI water when necessary. Some of the COIN samples (with a total silver concentration of 1.5 mM) were diluted ten times with 1 mM sodium citrate before measurement. FIGS. 10A and B show, respectively, the zeta potential measurements of silver particles of initial z-average size of 47 nm (0.10 M) with a suspending medium of 1.00 mM sodium citrate and the evolution of aggregate size (z-average) in the presence of 20 μM 8-aza-adenine.

Raman spectral analysis: for all SERS and COIN assays in solution, a Raman microscope (Renishaw, UK) equipped with a 514 nm Argon ion laser (25 mW) was used. Typically, a drop (50-200 μL) of a sample was placed on an aluminum surface. The laser beam was focused on the top surface of the sample meniscus and photons were collected for about 10-20 seconds. The Raman system normally generated about 600 counts from methanol at 1040 cm⁻¹ for a 10 second collection time. For Raman spectroscopy detection of an analyte immobilized on a surface, Raman spectra were recorded using a Raman microscope built in-house. This Raman microscope consisted of a water cooled Argon ion laser operating in continuous-wave mode, a dichroic reflector, a holographic notch filter, a Czerny-Turner spectrometer, and a liquid nitrogen cooled CCD (charge-coupled device) camera. The spectroscopy components were coupled with a microscope so that the microscope objective focused the laser beam onto a sample, and collected the back-scattered Raman emission. The laser power at the sample was about 60 mW. All Raman spectra were collected with 514 nm excitation wavelength.

Antibody Coating: A 500 μL solution containing 2 ng of a biotinylated anti-human antibody (anti-IL-2 or anti-IL-8) in 1 mM sodium citrate (pH 9) was mixed with 500 μL of a COIN solution (made with 8-aza-adenine or N-benzoyl-adenine); the resulting solution was incubated at room temperature for 1 hour, followed by adding 100 μL of PEG-400 (polyethyleneglycol-400). The solution was incubated at room temperature for another 30 min., then 200 μL of 1% Tween™-20 was added to the solution. The solution was centrifuged at 2000×g for 10 min. After removing the supernatant, the pellet was resuspended in 1 mL solution (BSAT) containing 0.5% BSA, 0.1% Tween-20 and 1 mM sodium citrate. The solution was then centrifuged at 1000×g for 10 min. The BSAT washing procedure was repeated for a total of 3 times. The final pellet was resuspended in 700 μL of diluting solution (0.5% BSA, 1× PBS, 0.05% Tween™-20). The Raman activity of the COINs was measured and adjusted to a specific activity of about 500 photon counts per μl per 10 seconds using a Raman spectroscope that generated about 600 counts from methanol at 1040 cm⁻¹ for 10 second collection time.

### EXAMPLE 2

Synthesis of COINs coated with BSA

Coating Particles with BSA: COIN particles were coated with an adsorption layer of BSA by adding 0.2% BSA to the COIN synthesis solution when the desired COIN size was reached. The addition of BSA inhibited further aggregation.

Crosslinking the BSA Coating: The BSA adsorption layer was crosslinked with glutaraldehyde followed by reduction with NaBH₄. Crosslinking was accomplished by transferring 12 mL of BSA coated COINs (having a total silver concentration of about 1.5 mM) into a 15 mL centrifuge tube and adding 0.36 g of 70% glutaraldehyde and 213 μL of 1 mM sodium citrate. The solution was mixed well and allowed to sit at room temperature for about 10 min. before it was placed in a refrigerator at 4° C. The solution remained at 4° C. for at least 4 hours and then 275 μL of freshly prepared NaBH₄ (1 M) was added. The solution was mixed and left at room temperature for 30 min. The solution was then centrifuged at 5000 rpm for 60 min. The supernatant was removed with a pipet leaving about 1.2 mL of liquid and the pellet in the centrifuge tube. The COINs were resuspended by adding 0.8 mL of 1 mM sodium citrate to yield a final volume of 2.0 mL.

FPLC Purification of Encapsulated COINS: The coated COINs were purified by FPLC (fast protein liquid chromatography) on a crosslinked agarose size-exclusion column. The concentrated COIN reaction mixture suspension (2.0 mL) was purified with a Superose 6 FPLC column on an AKTA Purifier. The COIN mixture was injected in 0.5 ml batches and an isocratic flow of 1 mM sodium citrate at 1 ml/min was applied to the column. Absorbance at 215 nm, 280 nm, and 500 nm was monitored for peak collection. The encapsulated COINs eluted at about 7-9 min., while the BSA/crosslinked BSA fraction eluted at about 9-11 min. Glutaraldehyde, sodium borohydride, and Raman labels eluted after about 20 min. Fractions from multiple FPLC runs were combined.

Antibody Conjugation: To attach an antibody probe to the encapsulated COINs, 500 μL of the encapsulated COIN solution was mixed with ⅕ volume of 10 mM EDC in water (EDC: 1.92 mg, water: 1 mL) to yield a final EDC concentration of 2 mM. The COIN-EDC reaction was allowed to proceed at room temperature for 15 min. Then, 500 μL of water was added and the solution was centrifuged at 5000 g for 10 min. The supernatant was removed and the residue was re-suspended in 1 mL of water and 50 μL of 1% Tween-20. The solution was centrifuged again at 5000 g for 10 min. and the supernatant was removed. 150 μL of water and 10 μl of 500 μg/mL detection antibody (to a final conc. about 0.2 μM) was added and the conjugation was allowed to proceed at room temperature for 30 min. with shaking every 10 min. 20 μL of PEG-400 was added to the reaction mixture. After 10 min., 500 μL of COIN wash solution (1 mM citrate, 0.5% BSA, 0.05% Tween-20) was added. After another 10 min., the mixture was centrifuged at 2000 g for 10 min., the supernatant was removed, and the pellet was washed 2 more times with COIN wash solution. The final COIN-ab product was re-suspended in 50 μL of 1 mM citrate and the Raman signal was measured. The product was stored at 4° C. until binding activity and protein assay were performed (usually within a week).

### EXAMPLE 3

Synthesis of Silver and Gold Particles coated with Silica

Silver COINs are coated with silica by adding 100 μL TEOS to a stirred solution of 75 mL 100% ethanol and 20 mL of a dilute COIN suspension created by adding about 1 to about 10 mL of a 1 mM COIN solution to deionized water. Then, 5 mL of a 28% NH₃ solution is added and the solution is maintained at room temperature with gentle stirring for 19 hours. The final molar composition of the coating solution is 4.6 mM TEOS and 0.82 M NH₃. At the end of 19 hours, the solution is diluted 4× with deionized water and centrifuged at 5000 rpm for 15 min. to remove the organic solvent. The suspending solution is replaced with 1 mM sodium citrate.

The above coating procedure was used to coat gold particles as well. Our results indicate that pretreatment with silane-based coupling reagents is not necessary to promote the formation of a silica coating.

What is claimed is:

1) A nanocluster of metal particles having a unique Raman signature, wherein the unique Raman signature is produced by at least one Raman active organic compound incorporated within the nanocluster, and wherein the Raman active organic compound is a molecule having a conjugated aromatic system comprised of at least two aromatic rings and at least two atoms selected from the group consisting of nitrogen and sulfur.

2) The nanocluster of claim 1 wherein the metal particles are comprised of a metal selected from group consisting of silver, gold, copper, palladium, platinum, and aluminum.

3) The nanocluster of claim 1 wherein the metal particles are comprised of silver or gold.

4) The nanocluster of claim 1 wherein the nanocluster has an average diameter of about 20 nm to about 200 nm.

5) The nanocluster of claim 1 wherein the nanocluster has an average diameter of about 50 nm to about 150 nm.

6) The nanocluster of claim 1 also comprising a surface layer selected from the group consisting of gold, silver, non-enzymatic globular or fibrous proteins, silica, block copolymers, soluble polymers, and organic thiol compounds.

7) The nanocluster of claim 1 also comprising a surface layer comprised of a protein selected from the group consisting of avidin, streptavidin, bovine serum albumen, transferrin, insulin, soybean protein, casine, gelatine, and mixtures thereof.

8) The nanocluster of claim 7 wherein the protein layer is crosslinked.

9) The nanocluster of claim 1 or 7 wherein the nanocluster is functionalized with a probe selected from the group consisting of antibodies, antigens, polynucleotides, oligonucleotides, receptors, carbohydrates, cofactors, and ligands.

10) A nanocluster of metal particles having a unique Raman signature, wherein the unique Raman signature is produced by at least one Raman active organic compound incorporated within the nanocluster and wherein the Raman active organic compound is selected from the group consisting of rhodamine, acridine orange hydrochloride, cresyl violet acetate, acriflavine neutral, dimidium bromide, 5,10,15,20-tetrakis(N-methyl-4-pyridinio)porphyrin tetra(p-toluenesulfonate), 5,10,15,20-tetrakis(4-trimethylaminophenyl)porphyrin tetra(p-toluenesulfonate), 3,6-diaminoacridine hydrochloride propidium iodide (3,8-diamino-5-(3-diethylaminopropyl)-6-phenylphenanthridinium iodide methiodide), trans-4-[4-(dimethylamino)styryl]-1-methylpyridinium iodide, 4-((4-(dimehtylamino)phenyl)azo)benzoic acid, succinimidyl ester, and mixtures thereof.

11) The nanocluster of claim 10 wherein the metal particles are comprised of a metal selected from group consisting of silver, gold, copper, palladium, platinum, and aluminum.

12) The nanocluster of claim 10 wherein the metal particles are comprised of silver or gold.

13) The nanocluster of claim 10 wherein the nanocluster has an average diameter of about 20 nm to about 200 nm.

14) The nanocluster of claim 10 wherein the nanocluster has an average diameter of about 50 nm to about 150 nm.

15) The nanocluster of claim 10 also comprising a surface layer selected from the group consisting of gold, silver, non-enzymatic globular or fibrous proteins, silica, block copolymers, soluble polymers, and organic thiol compounds.

16) The nanocluster of claim 10 also comprising a surface layer comprised of a protein selected from the group consisting of avidin, streptavidin, bovine serum albumen, transferrin, insulin, soybean protein, casine, gelatine, and mixtures thereof.

17) The nanocluster of claim 16 wherein the surface protein layer is crosslinked.

18) The nanocluster of claim 10 or claim 16 wherein the nanocluster is further functionalized with a probe selected from the group consisting of antibodies, antigens, polynucleotides, oligonucleotides, receptors, carbohydrates, cofactors, and ligands.

19) A method for detecting a known analyte in a sample solution, the method comprising:
contacting a sample solution containing an analyte with nanoclusters of metal particles having a unique Raman signature produced by at least one Raman active organic compound incorporated in the nanoclusters, wherein the Raman active organic compound is a molecule having a conjugated aromatic system comprised of at least two aromatic rings and at least two atoms selected from the group consisting of nitrogen and sulfur, and an attached probe specific for the known analyte under conditions;
contacting the sample containing the analyte with microspheres having an attached probe specific for the known analyte;
under conditions that allow the probes that are specific for the known analyte to complex to any known analyte present in the sample;
separating the microspheres in the solution from any uncomplexed nanoclusters;
detecting a Raman signal from a fluid solution containing a microsphere, wherein
detection of the Raman signature from a nanocluster is indicative of the presence of the analyte.

20) The method of claim 19 wherein the nanocluster has an average diameter of about 40 nm to about 200 nm.

21) The method of claim 19 wherein the nanocluster has an average diameter of about 50 nm to about 150 nm.

22) The method of claim 19 where the Raman active organic compound is selected from the group consisting of rhodamine, acridine orange hydrochloride, cresyl violet acetate, acriflavine neutral, dimidium bromide, 5,10,15,20-tetrakis(N-methyl-4-pyridinio)porphyrin tetra(p-toluenesulfonate), 5,10,15,20-tetrakis(4-trimethylaminophenyl)porphyrin tetra(p-toluenesulfonate), 3,6-diaminoacridine hydrochloride propidium iodide (3,8-diamino-5-(3-diethylaminopropyl)-6-phenylphenanthridinium iodide methiodide), trans-4-[4-(dimethylamino)styryl]-1-methylpyridinium iodide, 4-((4-(dimehtylamino)phenyl)azo)benzoic acid, succinimidyl ester, and mixtures thereof.

23) The method of claim 19 wherein the nanocluster has a bovine serum albumen coating and is comprised of at least one metal selected from the group consisting of copper, silver, gold, and aluminum.

24) The method of claim 19 wherein the probe is selected from the group consisting of antibodies, antigens, polynucleotides, oligonucleotides, receptors, carbohydrates, and ligands.

25) A method for distinguishing a plurality of biological analytes in a sample, the method comprising:
contacting a sample comprising a plurality of biological analytes with a set of Raman active metallic nanoclusters with each member of the set having a Raman signature unique to the set produced by at least one Raman active organic compound incorporated therein, wherein the Raman active organic compounds are a molecules having a conjugated aromatic system comprised of at least two aromatic rings and at least two atoms selected from the group consisting of nitrogen and sulfur, and having an attached probe specific for the known analyte, under conditions suitable to allow specific binding of probes attached to the set of metallic nanoclusters to analytes present in the sample to form complexes;
separating the bound complexes from any unbound complexes;
detecting Raman signatures from the complexed Raman active metallic nanoclusters,
wherein each Raman signature indicates the presence of the known biological analyte in the sample.

26) The method of claim 25 wherein the nanocluster has an average diameter of about 40 nm to about 200 nm.

27) The method of claim 25 wherein the nanocluster has an average diameter of about 50 nm to about 150 nm.

28) The method of claim 25 where at least one Raman active organic compound is selected from the group consisting of rhodamine, acridine orange hydrochloride, cresyl violet acetate, acriflavine neutral, dimidium bromide, 5,10,15,20-tetrakis(N-methyl-4-pyridinio)porphyrin tetra(p-toluenesulfonate), 5,10,15,20-tetrakis(4-trimethylaminophenyl)porphyrin tetra(p-toluenesulfonate), 3,6-diaminoacridine hydrochloride propidium iodide (3,8-diamino-5-(3-diethylaminopropyl)-6-phenylphenanthridinium iodide methiodide), trans-4-[4-(dimethylamino)styryl]-1-methylpyridinium iodide, 4-((4-(dimehtylamino)phenyl)azo)benzoic acid, succinimidyl ester, and mixtures thereof.

29) The method of claim 25 wherein the nanocluster has a bovine serum albumen coating and is comprised of at least one metal selected from the group consisting of copper, silver, gold, and aluminum.

30) The method of claim 25 wherein the probe is selected from the group consisting of antibodies, antigens, polynucleotides, oligonucleotides, receptors, carbohydrates, and ligands.

31) A method for the detection of a known cellular analyte, the method comprising:
contacting a sample containing a cellular analyte with a set of at least two composite organic inorganic nanoclusters, each member of the set having a Raman signature unique to the set produced by at least one Raman active organic compound incorporated in the nanoclusters, wherein the Raman active organic compounds are a molecules having a conjugated aromatic system comprised of at least two aromatic rings and at least two atoms selected from the group consisting of nitrogen and sulfur, and each member having an attached probe specific for a surface feature of the known cellular analyte;
separating the cellular analyte from any uncomplexed nanoclusters;
detecting a Raman signal from a solution containing the cellular analyte wherein the co-occurrence of at least two different unique Raman signatures is indicative of the presence of the known cellular analyte possessing at least one specific surface feature.

32) The method of claim 31 where at least one Raman active organic compound is selected from the group consisting of rhodamine, acridine orange hydrochloride, cresyl violet acetate, acriflavine neutral, dimidium bromide, 5,10,15,20-tetrakis(N-methyl-4-pyridinio)porphyrin tetra(p-toluenesulfonate), 5,10,15,20-tetrakis(4-trimethylaminophenyl)porphyrin tetra(p-toluenesulfonate), 3,6-diaminoacridine hydrochloride propidium iodide (3,8-diamino-5-(3-diethylaminopropyl)-6-phenylphenanthridinium iodide methiodide), trans-4-[4-(dimethylamino)styryl]-1-methylpyridinium iodide, 4-((4-(dimehtylamino)phenyl)azo)benzoic acid, succinimidyl ester, and mixtures thereof.

33) The method of claim 31 wherein each member of the set of nanoclusters has an attached probe specific for a different feature of the cellular analyte.

34) The method of claim 31 wherein the nanoclusters have an average diameter of about 40 nm to about 200 nm.

35) The method of claim 31 wherein the nanoclusters have an average diameter of about 50 nm to about 150 nm.

36) The method of claim 31 wherein the nanoclusters are comprised of gold or silver.

37) The method of claim 31 wherein the probes are selected from the group consisting of antibodies, antigens, receptors, carbohydrates, and ligands.

38) The method of claim 31 wherein the cell is fluorescently labeled and a fluorescence signal is detected.

39) A method for the detection of a known surface features present on a cellular analyte, the method comprising:
contacting a sample containing the cellular analyte with a composite organic inorganic nanocluster having a unique Raman signature produced by at least one Raman active organic compound incorporated in the nanocluster, wherein the Raman active organic compound is a molecule having a conjugated aromatic system comprised of at least two aromatic rings and at least two atoms selected from the group consisting of nitrogen and sulfur, and having an attached probe specific for a surface feature of the known cellular analyte;
fluorescently labeling the cellular analyte;
separating the cellular analyte from any uncomplexed nanoclusters;
detecting a Raman signal from a solution containing the cellular analyte wherein the co-occurrence of a fluorescence signal and a Raman signal is indicative of the presence of the known cellular analyte possessing at least one specific surface feature.

40) The method of claim 31 where at least one Raman active organic compound is selected from the group consisting of rhodamine, acridine orange hydrochloride, cresyl violet acetate, acriflavine neutral, dimidium bromide, 5,10,15,20-tetrakis(N-methyl-4-pyridinio)porphyrin tetra(p-toluenesulfonate), 5,10,15,20-tetrakis(4-trimethylaminophenyl)porphyrin tetra(p-toluenesulfonate), 3,6-diaminoacridine hydrochloride propidium iodide (3,8-diamino-5-(3-diethylaminopropyl)-6-phenylphenanthridinium iodide methiodide), trans-4-[4-(dimethylamino)styryl]-1-methylpyridinium iodide, 4-((4-(dimehtylamino)phenyl)azo)benzoic acid, succinimidyl ester, and mixtures thereof.

41) The method of claim 31 wherein each member of the set of nanoclusters has an attached probe specific for a different feature of the cellular analyte.

42) The method of claim 31 wherein the nanoclusters have an average diameter of about 40 nm to about 200 nm.

43) The method of claim 31 wherein the nanoclusters have an average diameter of about 50 nm to about 150 nm.

44) The method of claim 31 wherein the nanoclusters are comprised of gold or silver.

45) The method of claim 31 wherein the probes are selected from the group consisting of antibodies, antigens, receptors, carbohydrates, and ligands.
